(19) United States
(12) Patent Application Publication
(10) Pub. No.: US 2013/0237998 A1
(43) Pub. Date: Sep. 12, 2013

(54) SUTURING DEVICES AND METHODS WITH ABSORBABLE OR NON-ABSORBABLE MATERIAL INSERTS

(71) Applicants: Jeffrey M. Wallace, Saunderstown, RI (US); Peter J. Lukin, Norfolk, MA (US); Devang Shah, Wrentham, MA (US)

(72) Inventors: Jeffrey M. Wallace, Saunderstown, RI (US); Peter J. Lukin, Norfolk, MA (US); Devang Shah, Wrentham, MA (US)

(73) Assignee: C.R. BARD INC, MURRAY HILL, NJ (US)

(21) Appl. No.: 13/702,004

(22) PCT Filed: Jun. 10, 2010
(86) PCT No.: PCT/US10/01663

§ 371 Date: May 17, 2013

(51) Int. Cl. A61F 5/00

(52) U.S. Cl. CPC A61F 5/0083
USPC 606/144

(57) ABSTRACT

A method of promoting tissue adhesion to reinforce tissue apposition that may include collecting a first tissue portion, collecting a second tissue portion, placing one or more tissue securement devices through the first and second tissue portions, tightening the one or more tissue securement devices to approximate the first and second tissue portions, and placing a fixation agent between the first and second tissue portions. The method promotes tissue adhesion between one or more portions of tissue, wherein the tissue adhesion may reinforce a tissue apposition.

### FIELD OF INVENTION

This present invention relates to devices and methods for performing restriction or alterations within a body cavity that may lead to weight loss.

### BACKGROUND

Obesity, as defined by a body mass index (BMI) of 30 kg/m² or more, is a rapidly growing problem, currently affecting more than 30% of adults in the United States. Morbid obesity, as defined by a body mass index of 40 kg/m² or more or a BMI of 35 kg/m² or more in the presence of co-morbidities is also prevalent, affecting 3.1% of men and 6.7% of women. Obesity is commonly associated with many serious medical disorders including heart disease, diabetes, hypertension, hyperlipidemia, hypercholesterolemia, osteoarthritis and sleep apnea. In addition, approximately 300,000 adults in the U.S. die each year due to obesity-related causes.

The primary treatment objective for obese patients is weight reduction, which can improve co-morbid conditions and also reduces risk factors for disease. Even moderate weight loss (5%-10% of initial weight) produces health benefits and has been associated with marked reductions in the risk for the medical disorders listed above. While non-operative and pharmacologic weight loss therapies have met with only limited success, surgical intervention for morbid obesity, most frequently gastric bypass, is becoming increasingly common. However, the decision to undergo gastric bypass is a difficult one. Patients who choose to undergo gastric bypass are making a serious commitment to permanent life-style changes and are at risk for developing metabolic/nutritional complications resulting from the long-term malabsorptive effects of gastric bypass and food intake restriction. Long-term complications of gastric bypass including anemia secondary to iron or B₁₂ deficiency, mineral deficiencies (hypokalemia and hypomagnesia) and bone disease associated with secondary hyperparathyroidism are not uncommon. These conditions can be serious thereby necessitating lifelong medical follow-up to monitor for such events.

Although various procedures exist for the surgical treatment of morbid obesity, the Roux-en-Y gastric bypass (RYGB) has been identified as the gold standard for morbidly obese patients when non-invasive interventions have failed. The RYGB procedure entails the creation of a small gastric pouch to which the distal jejunum is attached via creation of an anastomosis referred to as a gastrojejunostomy (GJ). The procedure excludes more than 95% of the stomach, all of the duodenum and the proximal jejunum from digestive continuity. Weight loss is thought to result from reduced intake volume due to the small gastric pouch and limited GJ diameter, as well as from malabsorption due to the bypass of the proximal jejunum. The procedure is associated with a mean of 65-75% excess weight loss with 1% mortality and 10% morbidity.

Despite the favorable safety and effectiveness profile of the RYGB procedure, technical complications and inadequate weight loss may occur. Serious complications are not uncommon after open bariatric procedures. Adhesion formation may contribute to small bowel obstructions, which may require an additional operation for the patient. Incisional hernias are another complication associated with abdominal surgical procedures and have been shown to occur at a much higher rate after open gastric bypass surgery than after laparoscopic bypass surgery.

The significant morbidity associated with traditional weight loss surgery emphasizes the importance of the development of minimally invasive interventions that will result in patient weight loss, which may improve co-morbid conditions and also reduce risk factors for disease. Additionally, a minimally invasive or intragastrointestinal approach will minimize or eliminate many of the risks associated with open and laparoscopic procedures.

### BRIEF DESCRIPTION OF THE DRAWINGS

Certain embodiments of the invention are described with reference to the accompanying drawings, which, for illustrative purposes, are not necessarily drawn to scale.

FIG. 1 is a diagrammatic illustration of a distal end of an endoscope.

FIG. 2 is a partial sectional side view of a prior art endoscopic tissue apposition device.

FIG. 3 is a diagrammatic illustration of an embodied thread carrier where the suture material is fixated to a thread carrier.

FIG. 4 and FIG. 5 are partial sectional views of a prior art endoscopic tissue apposition device placing a suture through a fold of tissue.

FIG. 6 is an isometric transparent view of a prior art single intubation, multi-stitch endoscopic tissue apposition device.

FIG. 7 through FIG. 10 are illustrations of an endoscopic tissue apposition device placing a continuous suture pattern to accomplish tissue apposition.

FIG. 11 is an illustration of an endoscopic tissue apposition device placing an interrupted suture stitch to approximate two opposing sections of wall tissue together.

FIG. 12 is an illustration of a series of interrupted suture stitches placed in a pattern to accomplish tissue apposition.

FIG. 13 through FIG. 15 are illustrations of different suture site configurations that are possible to alter the volume, capacity, or function of the stomach.

FIG. 16 is an illustration of the application of a glue or a fixation agent used in conjunction with a tissue apposition device to reinforce a tissue apposition.

FIG. 17 is an illustration of the application of a biocompatible fabric to promote tissue bridging in conjunction with a tissue apposition device to reinforce a tissue apposition.

FIG. 18 is an illustration of the application of ablation to damage a portion of tissue to promote tissue bridging in conjunction with a tissue apposition device to reinforce a tissue apposition.

FIG. 19 is an illustration of the application of ablation to damage one or more portions of tissue, wherein the ablation is transmitted through a tissue securement device.

FIG. 20 is an illustration of the application of ablation to damage one or more portions of tissue, wherein the ablation is transmitted through a fixation agent.

FIG. 21 is an illustration demonstrating longitudinal and circumferential directionality.

FIG. 22 is an illustration of tissue securement device sites proximal to the pylorus and antrum.

FIG. 23 is an illustration that demonstrates stretching and/or compression of tissue resulting from a tissue apposition.

FIG. 24 is an illustration of an embodied fixation agent comprising fibers woven or knitted together.

FIG. 25 is an illustration of an embodied fixation agent comprising a sheet with one or more fenestrations within the sheet.

FIG. 26 is an illustration of an embodied fixation agent comprising multiple layers of material.

### DETAILED DESCRIPTION

The present invention provides methods for the apposition of tissue between two or more tissue surfaces. The embodied methods may be useful for external or internal tissue regions, but may be especially useful in endoscopic procedures. One example of such an endoscopic procedure is the endoscopic suturing of gastrointestinal tissue to reduce the volume, capacity, or function of the gastrointestinal cavity as a possible treatment for obesity. Another example of such an endoscopic procedure is the endoscopic suturing of gastrointestinal tissue to close or reduce a fistula. U.S. Pat. Nos. 4,841,888, 5,037,021, 5080,666, 5,792,153, and U.S. patent application Ser. No. 10/847,190 describe endoscopic suturing systems and methods with which the present invention is useful or may be used. Those patents and patent applications are incorporated by reference herein, in their entirety. A brief description of the basic elements of the endoscopic suturing systems and methods is presented below and the description of the illustrative embodiments will focus on the methods of the present invention as it is preferably used in endoscopic procedures.

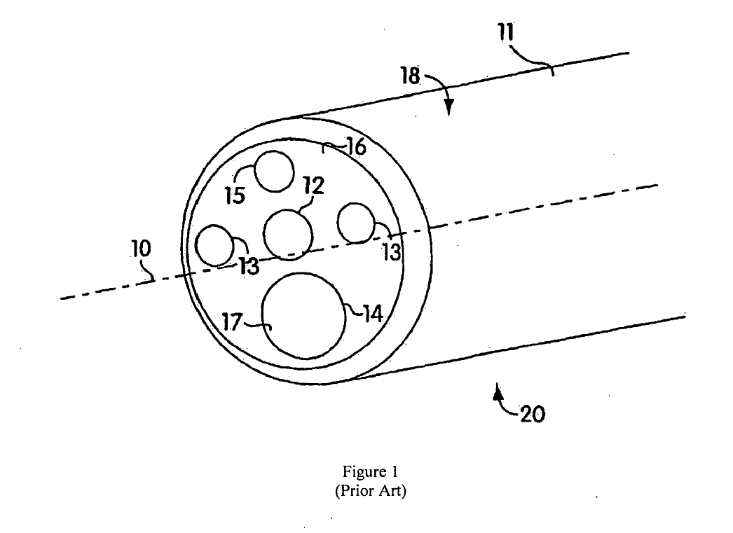

FIG. 1 shows the distal end 18 of a flexible endoscope 20 with which the present invention may be used. Terminating at a distal face 16 of the endoscope are several channels through which various functions may be performed. Typically, at least one large working channel lumen 14 is provided through which various medical instruments, catheters or accessory control mechanisms may be passed. In the case of viewing endoscopes, a viewing lens 12 is provided on the distal face of the endoscope to permit viewing via optical fibers or digital electronics that extend from the lens of the endoscope to its proximal end. Lights 13 illuminate the treatment site so that it may be viewed through the lens 12. Some endoscopes also have a fluid port 15 through which solution may be passed under pressure to rinse the lens of biological debris during a procedure. Additionally, a fluid port 15 may be used to transport fluid into the treatment site.

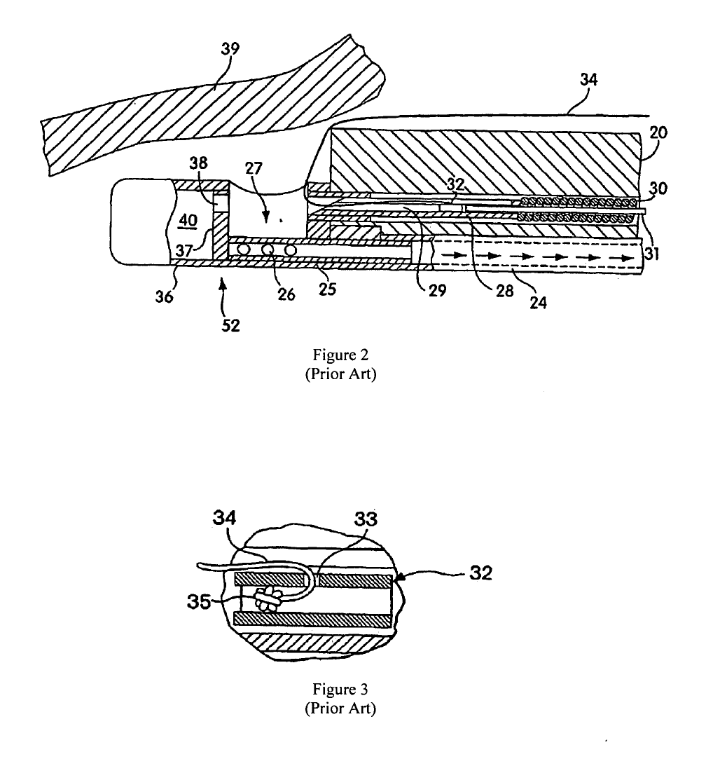

FIG. 2-4 depict a prior art endoscopic tissue apposition device disclosed in U.S. Pat. No. 5,792,153. FIG. 2 shows the distal end of a flexible endoscope 20, on which a sewing device 52 is attached. As mentioned above, the endoscope is provided with a viewing channel, which is not shown, but which terminates at a lens 12 on the distal face of the endoscope. The endoscope is further provided with a biopsy/working channel 14, and a suction channel 24, the proximal end of which is connected to a source of reduced pressure (not shown). The sewing device 52 has a tube 25, which communicates with the suction pipe 24 and may have a plurality of perforations 26 therein. These perforations communicate with an upwardly open cavity 27 formed in the sewing device that may be embodied as a vacuum chamber.

A hollow needle 28 is mounted in the biopsy channel 14, with its beveled tip extending into the sewing device. The needle has a channel 29 extending therethrough. A flexible, wire-wound cable 30 has its forward end attached to the rear of the needle 28, and a center wire 31 runs within the cable 30, along the entire length thereof, and is longitudinally movable with respect thereto. The diameter of the wire 31 is such that it is longitudinally movable within the channel 29 and, in the position shown in FIG. 2, the forward end portion of the wire 31 extends into the rear end portion of the channel 29.

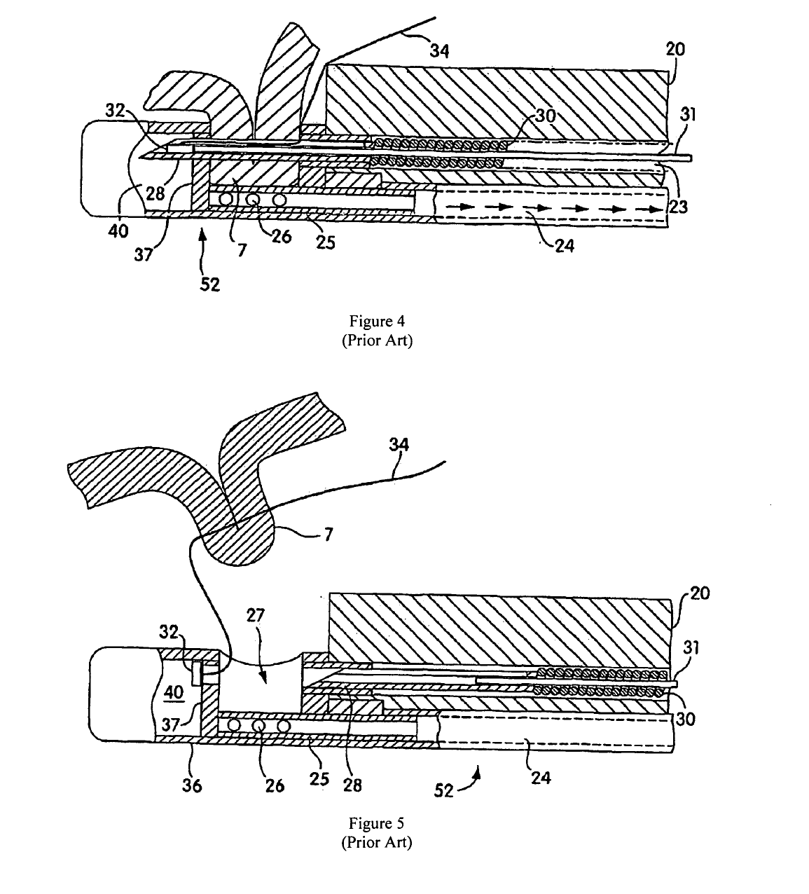

A thread carrier in the form of a tag 32 is mounted in the channel 29. The tag is shown in more detail in FIG. 3. The tag may be hollow and may have an aperture 33 extending through the side-wall thereof. As can also be seen in FIG. 3, one end of the thread 34 is secured to the tag by passing it through the aperture 33 and fixating the thread within the tag. One embodiment of the fixating the thread within the tag is illustrated in FIG. 3 by passing the thread through the aperture and tying in the end of a knot 35 of sufficient size to prevent the thread from escaping from the tag.

The sewing device has a hollow head portion 36 defining a chamber 40 therein, with the head portion 36 and the endoscope 20 being on opposite sides of the cavity 27. Between the chamber 40 and the cavity 47 is a wall 37, in which there is formed an aperture 58. The aperture 38 has a diameter that is marginally greater than the external diameter of the needle 28 and the aperture 38 must be sufficiently small to prevent tissue from being forced through the aperture and causing the needle to jam. Finally, FIG. 2 shows a portion of the patient's tissue 39, in which a stitch is to be formed.

In operation, suction is applied to the suction pipe 24, and hence, via the perforations 26 in the tube 25 to the cavity 27. This sucks into the cavity a U-shaped fold 7 of the tissue 39 as shown in FIG. 4. The hollow needle 28 is pushed through the U-shaped tissue fold 7 by exerting a distal (leftward) force on the center wire 31. After full advancement of the needle, the tip portion of the needle 28 is on the left-hand side of the wall 37, within the chamber 40 in the hollow head portion 36, and the tag 32, within the channel 29, lies to the left of the wall 37.

Continued distal movement of the wire 31 pushes the tag 32 out of the channel 29 and into the chamber 40. The wire 31 is then withdrawn proximally (rightwardly), followed by the proximal withdrawal of the cable 20, to bring both back to the positions which they occupy in FIG. 2. The suction is then discontinued allowing the U-shaped tissue fold 7 to be released from the cavity 27. The position is then as shown in FIG. 5. Finally, the endoscope and sewing device are withdrawn from the patient. In so doing, the thread 34 is pulled partially through the tissue fold 7, since the tag 32 is trapped in the chamber 40. The end result is that both ends of the thread are outside of the tissue and can be knotted and/or severed as may be appropriate. It should be noted that a multiple stitch embodiment is also disclosed in U.S. Pat. No. 5,792,153.

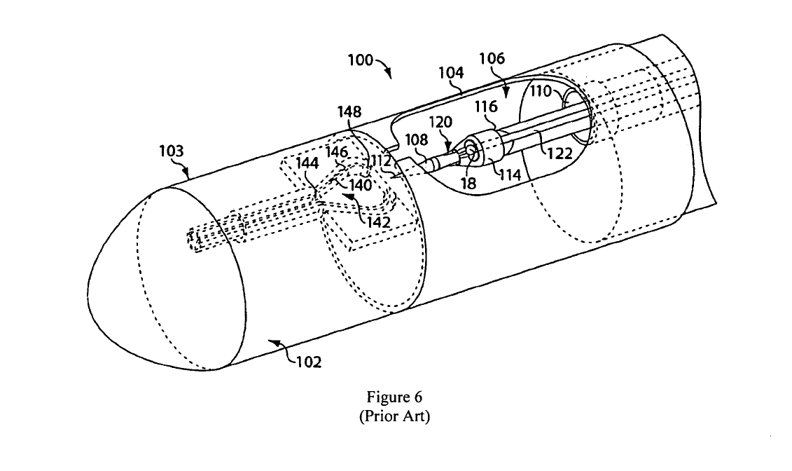

FIG. 6 depicts an embodiment of a prior art tissue apposition device capable of securing multiple tissue sites together with only one intubation of an endoscope carrying a suturing capsule at its distal end into the patient. A comprehensive discussion of the mechanisms associated with the tissue apposition device embodied in FIG. 6 is presented in pending U.S. application Ser. No. 10/847,190, incorporated by reference herein. The single intubation, multi-stitch endoscopic suturing system shown in FIG. 6, includes a suturing capsule 100 positioned at the distal end of an endoscope. The capsule is configured to receive a needle 108 slidable through a needle track 110 formed through the capsule. The needle may be a solid stainless steel shaft with a sharpened distal tip 112 and be joined at its proximal end to a pusher shaft (not shown) that extends proximally from the suture capsule, through the working channel of the endoscope. When the needle is moved longitudinally through the needle track, it traverses the suction chamber 106 so that tissue suctioned into the chamber will be penetrated by the distally advancing needle.

The needle 108 may carry an annular suture tag 114 that fits closely about the outside surface of the needle. Joined to the suture tag is one end of a suture 18 that will be carried through a suctioned tissue portion when the needle carrying the suture tag 114 is advanced distally. The suture tag is releasably and selectively secured to the outside surface of the needle by a suture tag lock 120. Full distal advancement of the needle places the suture tag 114 within the confines of a suture tag catch 140. After penetrating a captured tissue portion and entering the suture catch, the suture tag lock 120 may be released and the needle withdrawn proximally leaving behind the suture tag 114 in a nest area 142 of the suture tag catch. After capture and release of the suture tag into the suture tag catch 140, the needle may be withdrawn proximally and the tissue released from the suction chamber 106 with a suture 18 left passing through the tissue and having one end joined to the captured suture tag at the distal end 103 of the capsule and the other end of the suture extending into the needle track 110, through the working channel of the endoscope and exiting the proximal end of the endoscope.

The steps for retrieval of the tag are substantially the reverse of the steps illustrated for delivering the tag to the suture catch. Once the tissue is released from the capsule the tag may be recaptured by the needle in readiness for another stitch through either the same or a different captured tissue portion. By shuttling the tag and its associated suture through a series of captured tissue portions in this fashion, a plurality of stitches can be formed without requiring removal of the capsule for reloading.

Some embodiments described herein may utilize a vacuum chamber or suction to pull at least a portion of tissue within a chamber or within a path a tissue securement device may pass when it is deployed. In embodiments that comprise suturing as at least part of the tissue securement device, the suction or vacuum may pull the at least a portion of tissue within the path of a needle attached to the suture material such that the suture material may be advanced through said tissue portion. Other embodiments are certainly possible, wherein a mechanical grasper or mechanical device may be used to pull at least a portion of tissue within a chamber or within a path the tissue securement device may pass when it is deployed.

In embodiments comprise suturing or stitching as at least a portion of the tissue securement device, various embodiments of the invention are possible, wherein partial thickness stitches are placed. In other embodiments, full thickness stitches may be placed. Therefore, in embodiments which may comprise suturing where a tissue fold is collected by a tissue apposition device, one or more stitches placed by the tissue apposition device may be either partial thickness or full thickness stitches.

Using a preferred embodiment of a tissue apposition device, a plurality of methods are described below to appose and join internal tissue together, in a manner that may result in altering volume, capacity, or function of a body cavity. A body cavity may be defined as any opening or space within a patient's body that is accessible by endoscopic or laparoscopic devices. Examples of body cavities may include, but are not limited to, oral cavity, esophagus, stomach, small intestines, colon and rectum. A preferred embodiment of the invention utilizes a tissue apposition device within the stomach to alter the volume, capacity, or function of the stomach. By limiting the capacity of the stomach, a patient may not be able to eat as much food, thus potentially causing a reduction in the patient's food intake. This reduction in food intake may result in weight loss of the patient. Additionally, changes in at least a portion of stomach's function may result in an alteration of the patient's food intake, which may result in weight loss. Additional embodiments utilize a tissue apposition device to at least partially close or reduce one or more fistulas within the gastrointestinal tract.

The present embodiments of the invention may utilize one or more tissue securement devices to at least partially accomplish tissue apposition and joining of internal tissue.

Examples of tissue securement devices may include, but are not limited to, one or more suture materials, one or more staples, one or more magnets, one or more pins, one or more rods, or a combination thereof. A tissue securement device may comprise a combination of the aforementioned devices as well. One or more tissue securement devices may be comprised within a tissue apposition device, wherein the tissue apposition device may appose and join portions of internal tissue together.

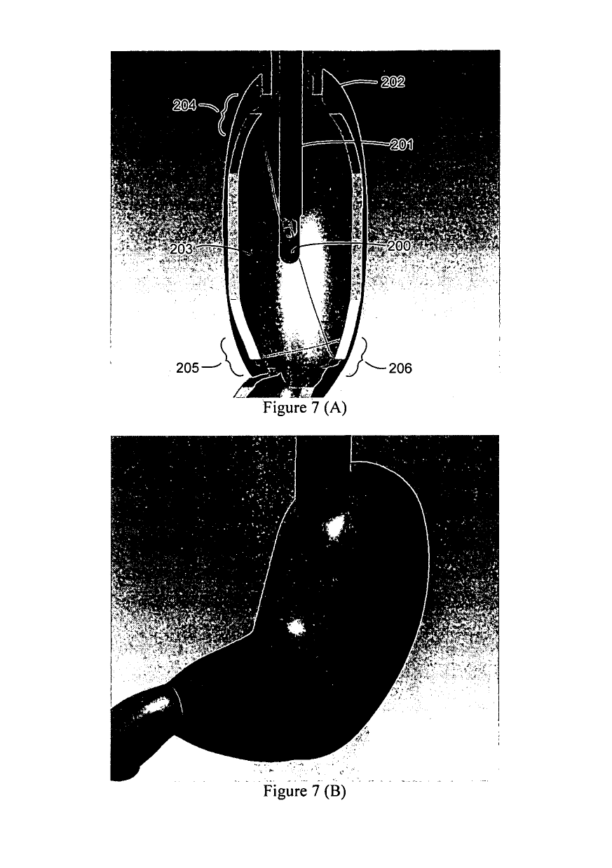

FIG. 7 through FIG. 10 illustrate an embodiment of the present invention utilizing suturing to alter the volume, capacity, or function of the stomach. In FIG. 7A, a tissue apposition device 200 that is mounted on an endoscope 201 is positioned within the lumen of the stomach 202. In this embodiment, the tissue apposition device places stitches into the substantial vicinity of a plurality of tissue sites (204, 205, 206), whereby a suture 203 passes through at least a portion of tissue within the substantial vicinity of the respective tissue sites. The tissue apposition device begins by first placing a tissue securement device into the tissue and passing the suture 203 through the tissue in the substantial vicinity of a first tissue site 204. The device is then navigated to a second tissue site 205 and a second stitch is placed passing the suture through tissue in the substantial vicinity of the second tissue site. Following this, the tissue apposition device may be repositioned to a third tissue site 206. A third stitch may be placed and the suture passed through the tissue in the substantial vicinity of the third tissue site. (The steps to place the stitches are not demonstrated in FIG. 7A) FIG. 7B illustrates an external illustration of the stomach as the tissue apposition device places stitches at the tissue sites 204, 205, 206.

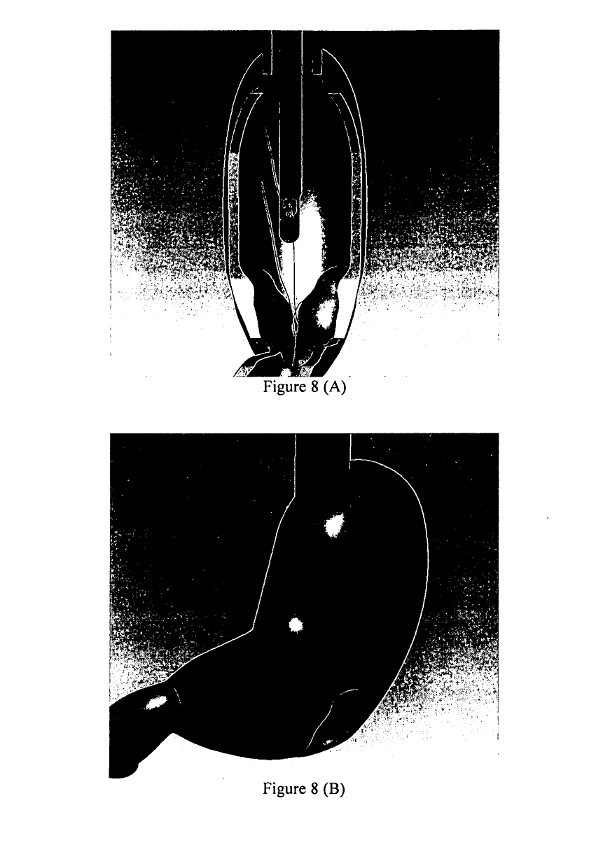

As the embodied tissue apposition device continues to place stitches at tissue sites, the tissue sites may reside on opposing walls of the cavity (for example, tissue sites 205 and 206 are on opposing walls). Through the embodied method, the opposing walls of the cavity may begin to approximate as the suture is drawn tight. The tissue approximation of opposing walls is illustrated in FIG. 8A. Furthermore, as the opposing walls are apposed together, the volume, capacity, or function of the body cavity may be altered. This is illustrated in FIG. 8B, where the tissue sites on opposing walls are being approximated when the suture is pulled tight.

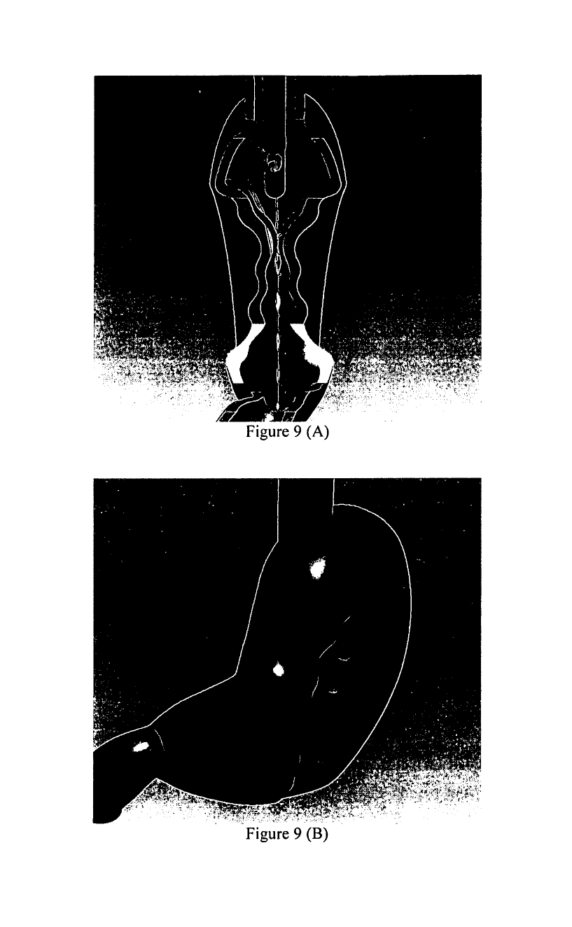

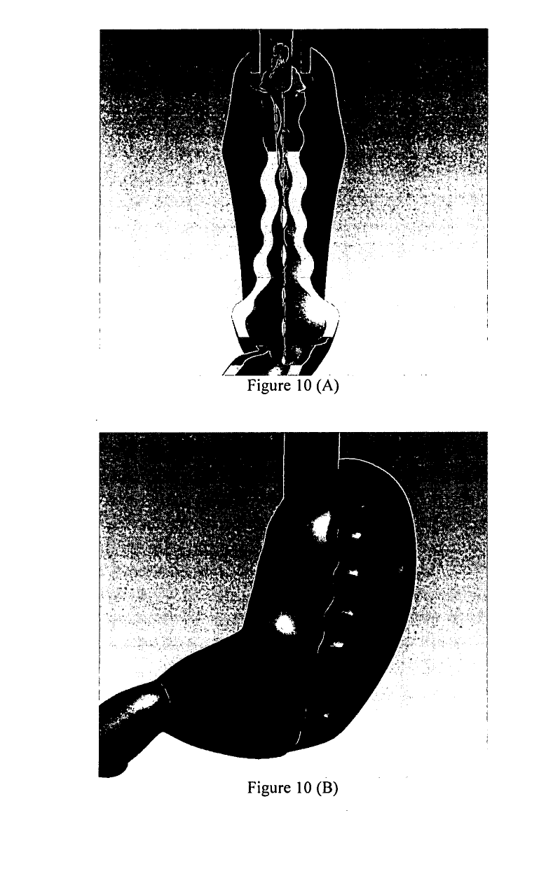

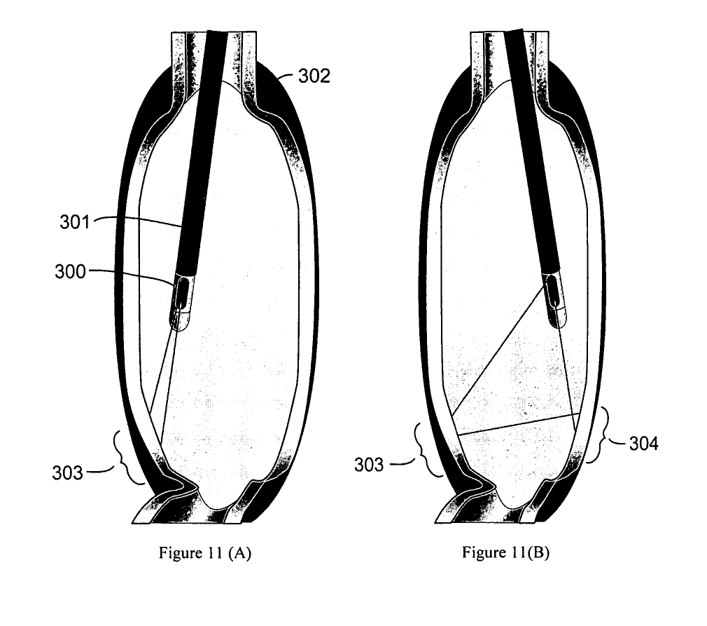

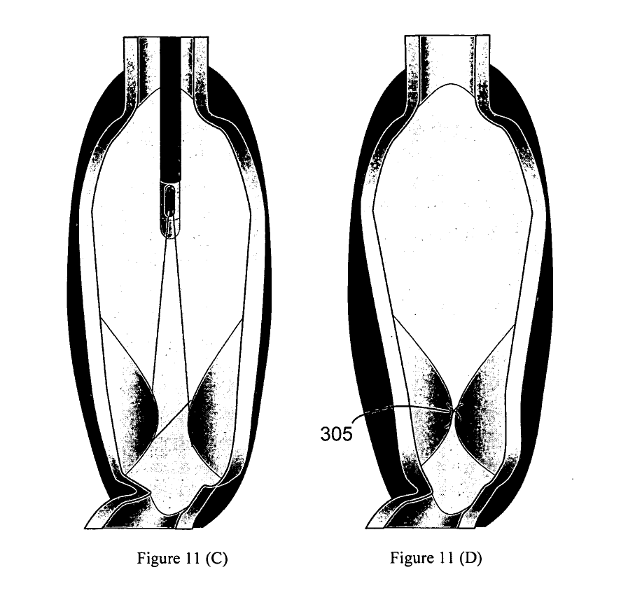

FIG. 9 and FIG. 10 illustrate a continuation of the tissue apposition process as the tissue apposition device sutures from a distal portion of the stomach to a proximal portion of the stomach. As more stitches are placed and more of the opposing cavity walls are approximated, the volume, capacity, or function may continue to be altered. FIG. 10 illustrates the state of the stomach after the embodied method has been completed. The tissue apposition was accomplished in a linear fashion in the direction of distal to proximal. After placing all the sutures as appropriate, the suture may be pulled tight and can be fixated with a method including, but not limited to, a knot or a suture lock device, following which the suture may be severed as may be appropriate.

The preferred embodiments illustrated in FIG. 7 through FIG. 10 demonstrate tissue apposition to alter the volume, capacity, or function of the stomach using a continuous suture stitch method. Other embodiments of the invention are also possible, including the use of different stitch methods. These stitch methods may include, but are not limited to, continuous, interrupted, and figure of eight stitches. Combinations of stitch methods are also possible.

FIG. 11 illustrates an alternate embodiment that may make use of interrupted stitches to approximate the walls of the cavity. The tissue apposition device 300, which is mounted on an endoscope 301 is positioned within a stomach cavity 302. As shown in FIG. 11A, the tissue apposition device is navigated to the distal portion of the stomach and a stitch is placed at a first tissue site 303 by passing the suture through tissue in the vicinity of the first tissue site 303. Following this, as shown in FIG. 11B, the tissue apposition device is repositioned to a second tissue site 304, preferable on an opposing wall of the cavity. At the second tissue site, a stitch is placed and the suture is passed through tissue in the vicinity of the second tissue site 304. The suture is then pulled tight, as illustrated in FIG. 11C, whereby the tissue sites approximate and appose. The suture may then be fixated 305 with a method including, but not limited to, tying a knot or applying a suture lock device, following which the suture may be severed as may be appropriate shown in FIG. 11D. If the tissue sites are located on opposing walls from one another, the tissue apposition may cause the walls to be pulled in, thereby altering the volume, capacity, or function of the body cavity. The embodied method utilized a tissue apposition device comprising suturing, but other embodiments utilizing other tissue securement devices are certainly possible.

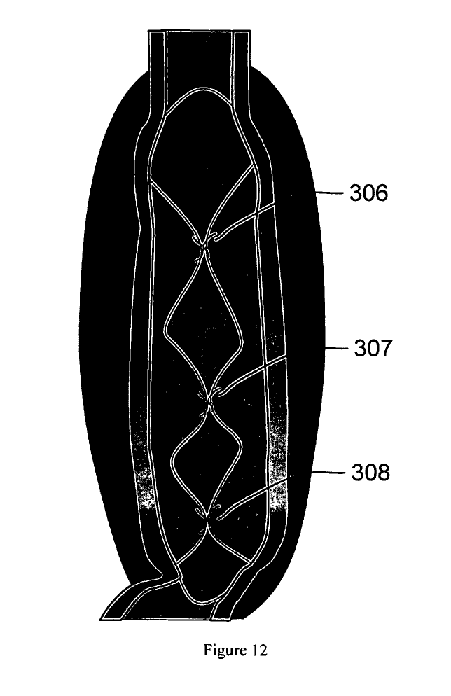

FIG. 12 demonstrates an embodiment of a series of suture stitches being placed in a manner akin to the embodied method illustrated in FIG. 11. Three suture stitches 306, 307, 308 are displayed in FIG. 12 for illustration purposes only, as a plurality of stitches may be possible. Stitches are preferably placed by the tissue apposition device in a series from a distal portion of the stomach to a proximal portion of the stomach. The total number of suture stitches that may be placed may vary based on one or more potential factors including, but not limited to the size of the stomach, the amount of cavity volume/function to be altered as appropriate, the location of the stitches, and the amount of tension caused by pulling the suture tight, or a combination thereof.

In preferred embodiments of the invention, tissue apposition may be accomplished by a series of linearly placed tissue securement devices as illustrated in FIG. 10, FIG. 11, and FIG. 12 (which utilize suture stitches as a tissue securement device). While the embodiments described herein demonstrate the placement of tissue securement devices in a distal to proximal linear direction, other embodiments are certainly possible. Tissue securement devices may be placed in different series, such as laterally across the stomach or in patterns, such as a zig-zag fashion to create an alteration of the volume, capacity, or function of the body cavity. Additionally, the location or distance between tissue securement devices can be varied as appropriate. In some embodiments, the distance between tissue securement devices may be very small. In such embodiments, the series of suture stitches may approximate and appose the tissue to form a partition that prevents or partially limits the passage of matter through the partition. Such partitions may prevent or partially limit the passage of food particles through the partition. Other embodiments may deploy tissue securement devices in a pattern that when the tissue is apposed together to form a partition, the partition prevents or partially limits the passage of liquids through the partition.

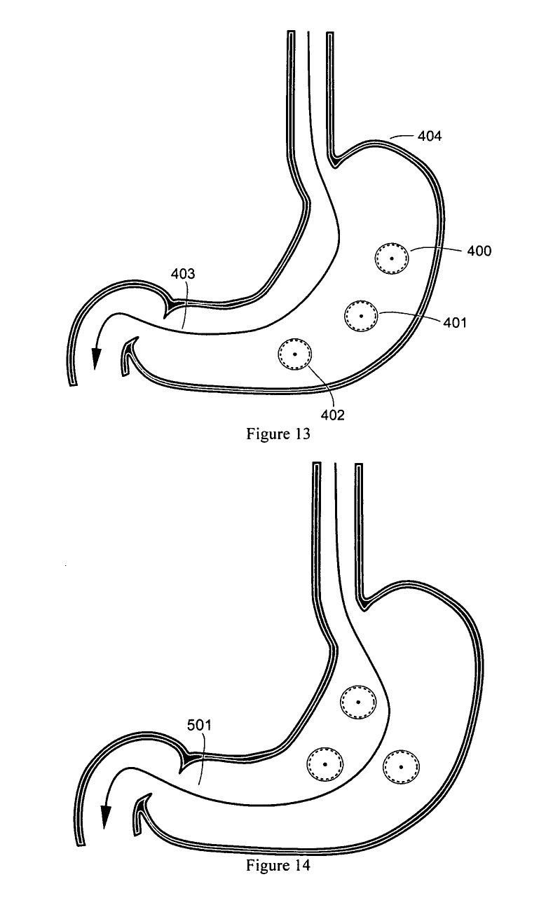

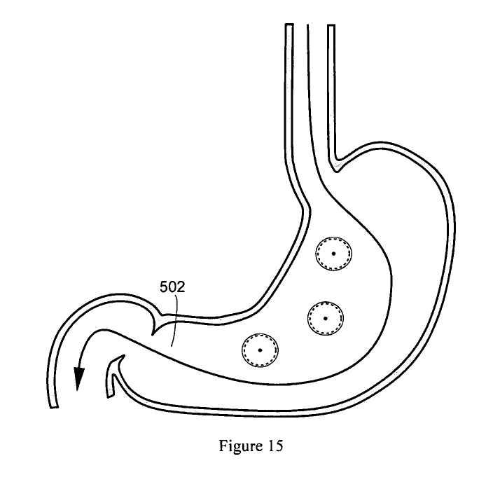

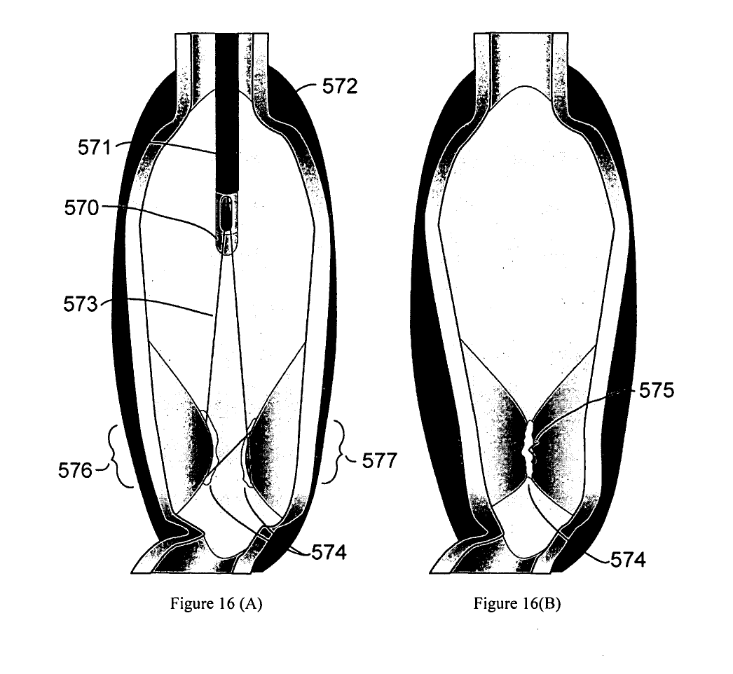

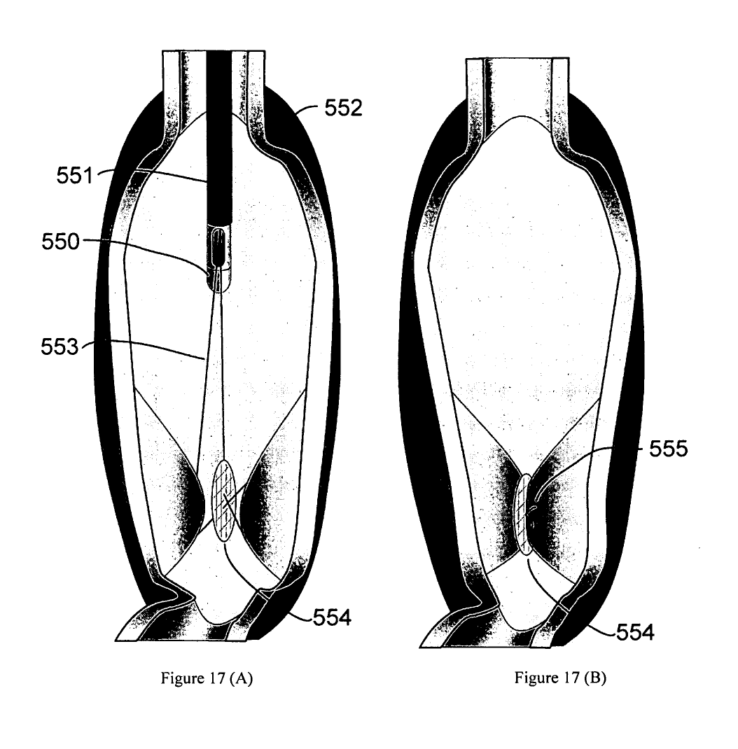

Further embodiments may include the deployment of at least one tissue securement device in a non-linear series. In such embodiments, the devices, such as a suture material, may be placed at a plurality of locations. The tissue securement devices may also be placed in clusters in locations or individually in locations within the cavity. In such embodiments, the tissue securement devices may approximate and appose the opposing walls together to alter the volume, capacity, or function of the stomach. FIG. 13 demonstrates one possible configuration. Three sites 400, 401, 402 are shown in FIG. 13 for illustrative purposes only, as a plurality of sites are possible. A single tissue securement device or a plurality of tissue securement devices may be placed at each site to approximate tissue. FIG. 14, FIG. 15 and FIG. 22 illustrate alternate embodiments of placing such sites. Again, a few tissue securement device sites are illustrated, but a plurality of tissue securement device sites may be used as appropriate. The arrows 403, 501, 502, 503 represent one possible pathway particles, such as stomach contents, may pass through the body cavity in the respective embodiments.

Embodiments of the present invention may include methods of promoting tissue adhesion between one or more portions of tissue to potentially reinforce tissue apposition sites. Some embodiments may utilize a tissue apposition device. In some embodiments, a plurality of tissue sites may be secured together by passing a tissue securement device, such as suture material, through each tissue site, tightening the securement device, and securing the tissue securement device. In embodiments that may use suture material, securing the tissue securement device may include, but are not limited to, a knot or a suture lock device. When the securement device is tightened, at least a portion of the tissue sites in which the securement device passes through will be approximated and may be placed in contact with some or all of the other tissue sites which are being approximated. The secured tissue securement device will maintain the tissue approximation. In some instances, however, the securement device may migrate or tear through the tissue over time, causing the tissue apposition to weaken or possibly fail. The amount of time the tissue securement device may maintain the tissue approximation varies on factors including, but not limited to, the individual patient, the depth the tissue securement device passes through the tissue, the physical properties of the tissue securement device, the consistency of the tissue, the tension on one or more tissue securement devices caused by the tightening one or more tissue securement devices, and the dynamic environment of the tissue and the body cavity. Therefore, multiple embodied methods are possible to strengthen the tissue apposition and reinforce the tissue approximation.

Embodiments of the present invention may include the use or presence of a fixation agent as part of a method to reinforce or strengthen a tissue approximation. This may include the placement of a fixation agent between a plurality of tissue sites, either before, during, or after the tissue approximation is secured. One such fixation agent may be, for example, a glue that is applied to at least one portion of the tissue sites that come into contact with other tissue sites within the tissue approximation. Fibrin glue is one example of a glue that may act as a fixation agent.

FIG. 16 presents an illustration of an embodiment that used a glue as a fixation agent in conjunction with suturing. In FIG. 16, an interrupted suture stitch pattern is placed in a manner similar to FIG. 11, although many other stitch patterns are possible. After the tissue apposition device 570, which is mounted on a endoscope 571, has passed the suture material 573 through at least one tissue site, a fixation agent, such as a glue 574, may be applied to one or more of the at least one tissue sites. While FIG. 16A demonstrates only two tissue sites 576, 577 for illustrative purposes, the embodiments of the present invention may comprise a plurality of tissue sites.

Following the deployment of the fixation agent, the suture material is pulled tight and the tissue sites are approximated and at least a portion of the tissue site 576 comes into contact with at least a portion of other tissue site 577. The suture material may be secured by methods including, but not limited to, a knot or a suture lock, following which the suture material may be cut as appropriate, the results of which are illustrated in FIG. 16B. The two sites are now approximated and secured with the suture material. In the case where the fixation agent requires activation, the fixation agent may then be activated or begin the activation process that will promote tissue adhesion to reinforce the tissue apposition. When the fixation agent is embodied as a glue, the glue may cure and bind the portions of the tissue site 576 that come into contact with other portions of tissue site 577. It is, of course, understood that various aspects of the present invention will be apparent to those skilled in the art. For example, the glue may be applied to only a portion of tissue or all the tissue that comes into contact with the other tissue. Additionally, the glue may be applied to a portion or all of the tissue sites prior to the tissue apposition device passing the suture through one or more tissue sites or the glue may be applied after tissue apposition has occurred.

Certain embodiments of the present invention may use a fixation agent that promotes tissue adhesion to reinforce tissue apposition, where the fixation agent is adapted to promote tissue adhesion through tissue growing, healing, or scarring. A tissue adhesion may be formed between a plurality of tissue sites when one or more portions of the plurality of tissue sites grow tissue that connects and/or binds with one or more other portions of the plurality of tissue sites. Having one or more portions of one or more tissue sites fusing together with one or more other portions of one or more tissue sites through a growing, healing or scarring process may be referred to as tissue bridging. Embodiments of the present invention may comprise a fixation agent adapted to promote tissue bridging between two or more tissue portions, which may reinforce tissue apposition and/or securement.

One embodiment of a fixation agent that promotes tissue adhesion may be one or more chemicals or substances that may act as a tissue growth factor. Examples of such chemicals or substances may include, but are not limited to, connective tissue growth factor (CTGF), vascular epithelial growth factor (VEGF), and tissue formation growth factor. The application of one or more chemicals or substances that may act as a tissue growth factor to one or more portions of tissue that are at least partially apposed together may accelerate, stimulate, or promote cellular growth between the one or more portions or tissue. This cellular growth may promote tissue bridging that may reinforce or strengthen a tissue apposition. Additionally, such chemicals or substances may accelerate, stimulate, or promote a healing or scarring process between the one or more portions of tissue that may be apposed.

One embodiment of a fixation agent that may facilitate tissue bridging between two or more portions of tissue is a body of biocompatible fabric. Such a biocompatible fabric may include a plurality of interstices which may be constructed or arranged to facilitate tissue infiltration and/or tissue bridging. The plurality of interstices may adapt the biocompatible fabric to allow tissue to infiltrate the fabric, which may act as a structure to promote new tissue development. A tissue apposition, where two or more portions of tissue are approximated and secured with a tissue apposition device, may comprise the biocompatible fabric with a plurality of interstices, whereby the plurality of interstices promote tissue bridging between two or more portions of tissue. The tissue bridging may reinforce the tissue apposition, wherein the reinforcement may increase the tissue apposition's resistance to being separated. The biocompatible fabric may be embodied by apparatuses including, but not limited to, a mesh of polypropylene monofilament or a mesh of PTFE monofilament.

FIG. 17 illustrates one embodiment of the present invention that comprises a biocompatible fabric with a plurality of interstices secured between two or more portions of tissue within a tissue apposition. A tissue apposition device 550 is mounted on an endoscope 551, which can be navigated within the lumen of stomach 552. A tissue securement device, illustrated in FIG. 17 as a suture stitch, is placed in a manner similar to FIG. 11, although many other securement devices are possible. After the tissue apposition device 550 has passed the suture material 553 through a plurality of tissue sites, where the tissue sites may reside at least partially on opposing walls of the stomach from one another, the biocompatible fabric 554 may be fixated or secured into the tissue apposition. FIG. 17A demonstrates two portions of tissue comprised in the tissue apposition for illustrative purposes only, as a plurality of tissue sites are possible. After the biocompatible fabric 554 is incorporated into the tissue apposition, the suture material is pulled tight and the tissue sites are approximated. At least a portion of the tissue sites may come into contact with the biocompatible fabric. The suture material may be secured by methods described herein, following which the suture material may be cut as appropriate, resulting in what may be illustrated in FIG. 17B. Portions of tissue in contact or in the vicinity of the biocompatible fabric may undergo a tissue healing, growing, and/or scarring process and infiltrate the interstices of the biocompatible fabric. The tissue infiltration of the fabric may facilitate or undergo tissue bridging between two or more portions of tissue, which may create a tissue adhesion that will reinforce the tissue apposition.

One embodiment of a fixation agent that may facilitate tissue bridging between two or more portions of tissue is a body of resorbable or absorbable material. By using a body of resorbable material, said body, when place within an in vivo environment, may be colonized by fibroblasts and revascularized. Examples of such a resorbable material may include, but is not limited to, animal or human collagen (especially porcine), animal or human intestinal sub-mucosal membrane, animal or human vesical sub-mucosal membrane, animal or human pericardium (especially bovine), portions of animal or human dermis, and/or a combination thereof. Said body of resorbable material may be either of human, animal, synthetic origin or a combination thereof. Such an embodiment may be placed between two or more portions of tissue, wherein the two or more portions of tissue may infiltrate or resorb one or more portion of the resorbable body into the tissue. The infiltration or incorporation of the fixation agent may promote tissue adhesion or tissue bridging between the two or more portions of tissue.

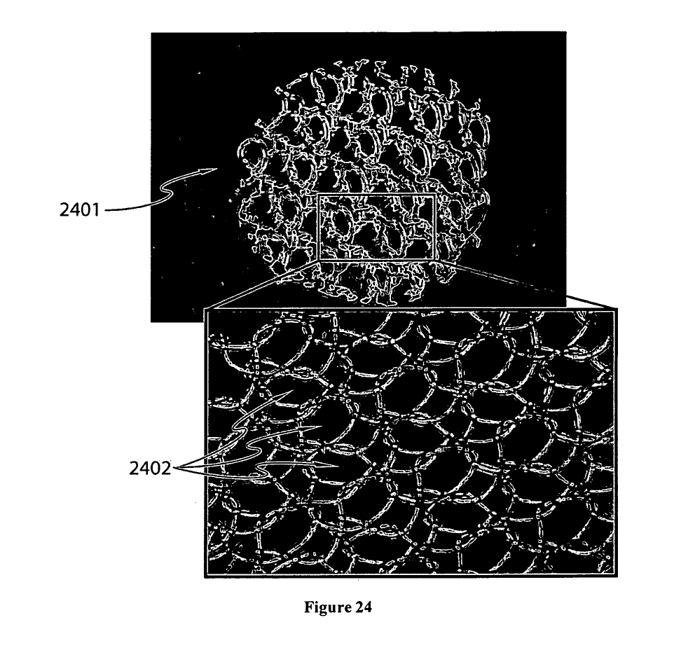

Embodiments of a fixation agent may comprise of fabric of resorbable or absorbable material with a plurality of interstices. FIG. 24 demonstrates one such embodiment, where a resorbable or absorbable fabric (2401) comprises one or more resorbable or absorbable fibers woven or knitted to allow a plurality of interstices (2402). When the fabric (2401) is placed between and comes into contact with two or more portions of tissue, the portions of tissue in contact or in the vicinity of the fabric (2401) may undergo a tissue healing, growing, and/or scarring process that my cause the tissue to infiltrate the interstices (2402) of the fabric (2401). The tissue infiltration may facilitate or undergo tissue bridging between two or more portions of tissue, which may result in tissue adhesion that reinforces the tissue apposition. Preferable dimensions of this embodiment would be a circular or elliptical shape where the greatest dimension across the fabric (i.e. diameter in the case of a circular pattern) would be 0.375-0.625 inches.

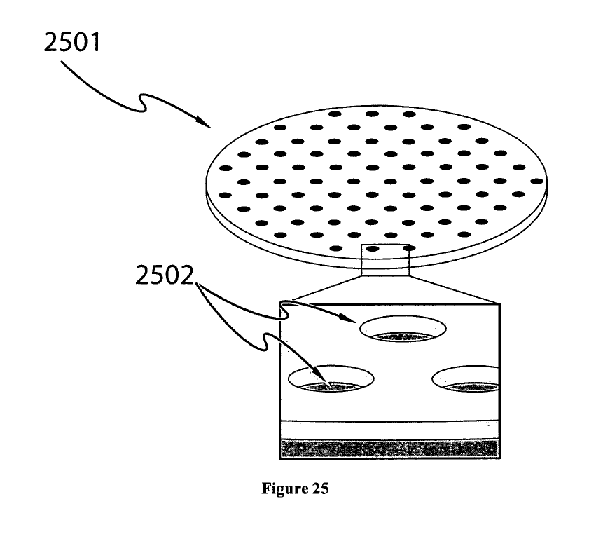

FIG. 25 illustrates another embodiment of a fixation agent comprising resorbable or absorbable material. In the embodiment, a sheet of one or more layers of resorbable material (2501) comprises one or more fenestrations (2502) within the sheet. The thickness of the sheet (2501) may preferably be between 0.5 and 1.0 mm in dimension. When the fenestrated sheet (2501) is placed between and comes into contact with two or more portions of tissue, the portions of tissue in contact or in the vicinity of the sheet (2501) may undergo a tissue healing, growing, and/or scarring process that my cause the tissue to infiltrate the fenestrations (2502) of the sheet (2501). The tissue infiltration may facilitate or undergo tissue bridging between two or more portions of tissue, which may result in tissue adhesion that reinforces the tissue apposition.

Embodiments of the invention that comprise resorbable or absorbable material may be processed, treated, or in combination with other materials in order to speed up or slow down the resorbtion or absorption into the body. One example of such a process would be to cross-link the individual fibers or components of the material. As such, the material may be engineered to maintain structural integrity for an approximate amount of time when the material is secured within a tissue apposition. Another example would be to coat a first material with a secondary material, wherein second material is a non-degradable or slowly degradable material. Therefore, the structural integrity of the first material would be preserved until such time that the second material had degraded enough for the first material to be exposed to the in vivo environment, thereby increasing the overall time it would take to degrade the coated material. A preferred amount of time for a resorbable or absorbable material to maintain its structural integrity might be approximately 12-14 weeks—which would allow for sufficient time for the tissue to complete its healing response and tissue bridging to be formed.

Some embodiment of the invention may comprise one or more layers, wherein the layers may be chosen from a list comprising: biocompatible non-absorbable synthetic materials, bioabsorbable or biodegradable synthetic materials (including but not limited to polymer structures), non-absorbable materials derived from natural sources, and bioabsorbable or biodegradable materials derived from natural sources. In certain embodiments, one or more layers may be constructed by laminating one or more materials together. In certain embodiments, one or more layers may be comprised of a first material coated with a second material.

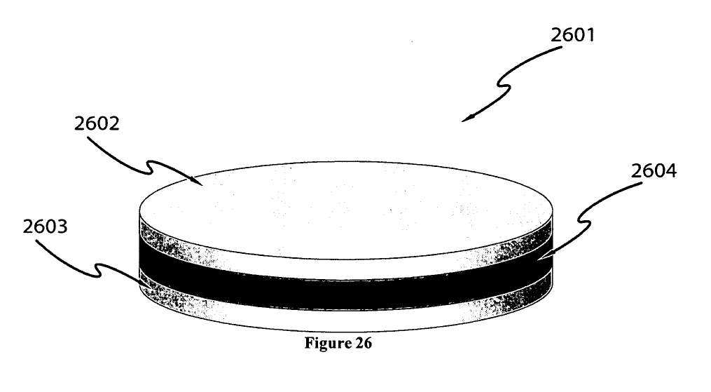

An embodiment of the present invention is illustrated in FIG. 26. In this figure, the embodiment comprises a fixation agent (2601) with three layers: a first layer (2602), a second layer (2603), and a middle layer (2604). The first and second layers may be chosen from a bioabsorbable or biodegradable synthetic material or a bioabsorbable or biodegradable material of natural origin. The middle layer may be chosen from a non-absorbable synthetic material or a non-absorbable material derived from natural sources. The fixation agent may be incorporated into a tissue apposition by being placed between and/or comes into contact with two or more portions of tissue. In certain embodiments, the combination of the three layers may provide mechanical strength that assist in placing the fixation agent within a tissue apposition (i.e. easier to manipulate). In certain embodiments, the first and second layers may protect or shield the middle layer during placement of the fixation agent into a tissue apposition. After the plication is placed, the first and second layers may absorb or degrade. This absorption or degradation may promote tissue infiltration into the middle layer and promote tissue bridging between tissue portions. the portions of tissue in contact or in the vicinity of the fixation agent (2601) may undergo a tissue healing, growing, and/or scarring process that my cause the tissue to infiltrate the fenestrations.

Other combinations of layers is possible in embodiments of fixation agents. For example, an embodiment may comprise of three layers, wherein an absorbable or resorbable material layer is sandwiched between two non-absorbable material layers. Another embodiment of a fixation agent may comprise two layers wherein one layer is chose from an absorbable or resorbable and the other layer is chosen from a non-absorbable material. Other embodiments may comprise of three or more layers, wherein a portion of the layers are chosen from absorbable or resorbable material and another portion of layers are chosen from non-absorbable materials.

Absorbable, resorbable, or degradable materials may include absorbable polymers such as polyglycolic acid (PGA), polylactic acid (PLA), polycaprolactone, and polyhydroxyalkanoate, or copolymers thereof (e.g., a copolymer of PGA and PLA); or tissue based materials (e.g., collagen or other biological material or tissue). It may also include polyanhydride, polyorthoester, or polyglycolic acid, alone or coated with extracellular components such as collagen, fibronectin, laminin, and complex mixtures.

In other examples, absorbable, resorbable, or degradable materials may include sheets of material that are built up from polymer fibers around a porous polymer core or an acellular biological tissue. Suitable synthetic polymers include bioerodable polymers, such as polyglycolic acid (PGA), polylactic acid (PLA), polyhydroxyalkanoate (PHA) and poly-4-hydroxybutyrate (P4HB), polycaprolactones, poly(lactide-co-glycolide) (PLGA), polycarbonates, polyamides, polyanhydrides, polyamino acids, polyorthoesters, polyacetates, polycyanoacrylates and degradable polyurethanes, and non-erodable polymers, such as polyacrylates, ethylene/vinyl acetate polymers and other substituted cellulose acetates and derivatives thereof.

Absorbable, resorbable, or degradable materials may further include materials derived of tissue extracellular matrix such as protein materials, such as collagen or fibrin, and polysaccharidic materials, like chitosan or glycosaminoglycans (GAGs).

In certain embodiments, the absorbable, resorbable, or degradable materials may comprise protein containing substances. This will enable degradation of the material by proteolytic enzymes. Such materials are preferably made of proteins such as collagen, keratin, fibrin, elastin, laminin, vimentin, vitronectin, fibronectin, fibrinogen and derivatives of these and the like or denaturated proteins such as gelatin.

In another embodiment, the absorbable, resorbable, or degradable material may comprise carbohydrate/polysaccharide containing substances. This may enable degradation by hydrolysis and enzymatic degradation of the polysaccharides. Such materials are preferably made of polysaccharides such as heparan sulfate, chondroitin sulfate, dermatan sulfate, heparin, keratan sulfate and derivatives of these, alginates, HSC cellulose and cellulose derivatives (CMC), some alginates, chitosan, chitin, pectin and pectin derivatives, hyaluronic acid and proteoglycans (mucopolysaccharides) and derivatives of these. Other absorbable, resorbable, or degradable materials are certainly possible.

Non-absorbable or non-degradable materials may include non-absorbable polymers such as polypropylene, polyethylene, polyethylene terephthalate, polytetrafluoroethylene, polyaryletherketone, nylon, fluorinated ethylene propylene, polybutester, and silicone, or copolymers thereof (e.g., a copolymer of polypropylene and polyethylene). Other non-absorbable or non-degradable materials are certainly possible.

Additional embodiments of the present invention comprise methods and/or techniques to promote tissue adhesion to reinforce tissue apposition. Certain embodied methods may be, for example, promoting tissue bridging between one or more portions of tissue, whereby the tissue bridging is instigated as a result of a tissue healing process, a tissue growing process, or a tissue scarring process. By causing a tissue injury or tissue damage to one or more portions of tissue, the body's regenerative healing process may be enabled to undergo tissue bridging between the one or more portions of tissue.

There are many methods of damaging portions of tissue or causing tissue injury that may fall within the scope of various embodiments of the present invention. Examples of causing tissue damage include, but are not limited to, the application of electrical energy, the application of one or more chemical substances, the application of thermal ablation, the application of cryo ablation, and the application of mechanical abrasion. Additionally, examples of causing tissue damage may include the application of laser energy onto at least a portion of the tissue or the application of argon plasma onto at least a portion of the tissue.

Several embodiments may incorporate the application of electrical energy. Electrical energy may include radiofrequency energy (either monopolar or bipolar). The electrical energy, when applied to tissue, may ablate the mucosal and possibly the submucosal layers. Following the tissue ablation, a tissue healing or scarring process will begin to at least partially regenerate the damaged tissue. Such tissue healing or scarring processes may be adapted to promote tissue bridging.

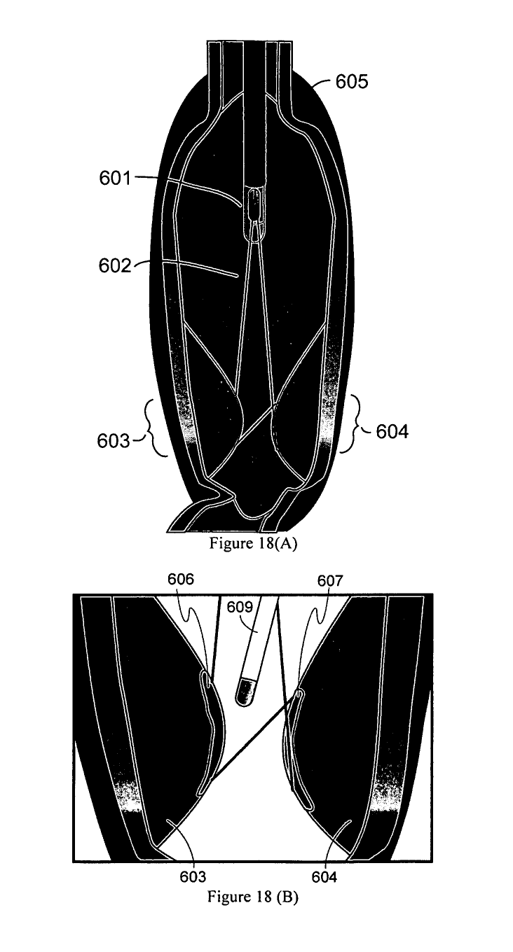

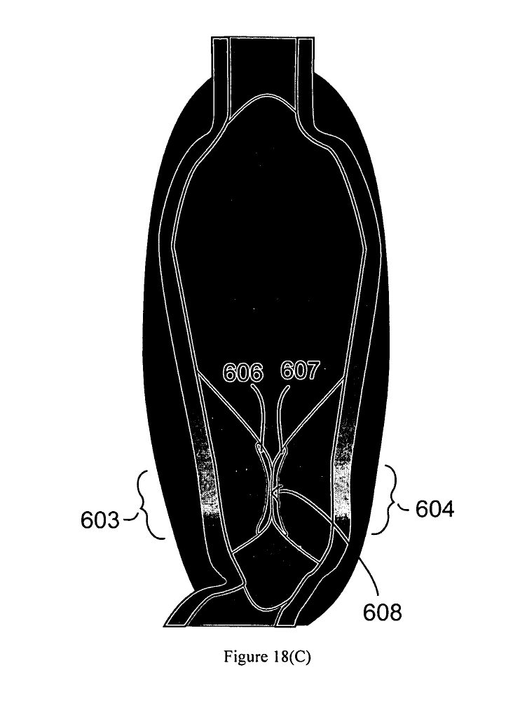

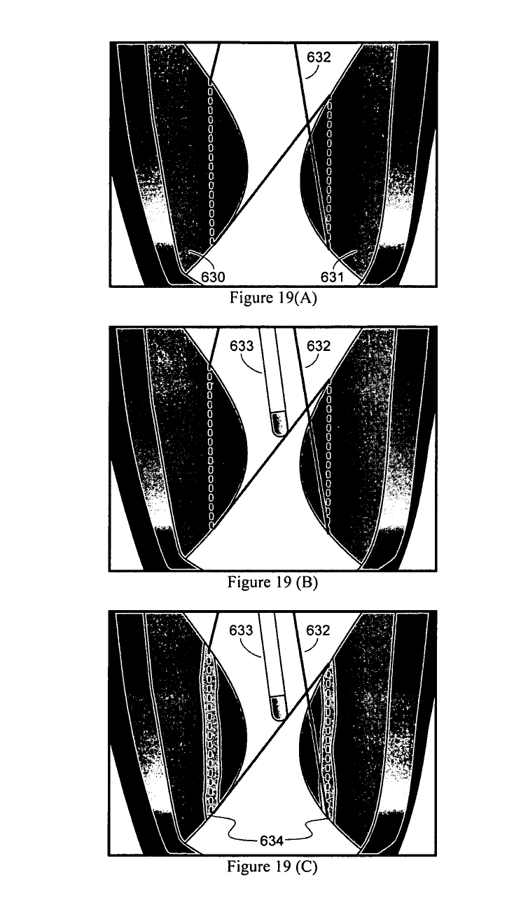

FIG. 18 demonstrates one possible embodiment that adapts electrical energy to ablate tissue, whereby the ablation promotes tissue bridging. Using a tissue apposition device 601 (as shown in FIG. 18(A)), a tissue securement device 602 (embodied in this illustration as suturing) is advanced through a first and second portion of tissue (603 and 604 respectively) in a manner that may be similar to that shown in FIG. 11. Tissue ablation may be accomplished through electrical energy. An electrocautery catheter or a similar device 609 is positioned within the body cavity 605 and is navigated into the vicinity of the first and second portions or tissue 603, 604, as illustrated in close up view in FIG. 18(B). By applying the electrical energy via the electrocautery catheter to the surface of the first and second portion of tissue, the mucosa and possibly the submucosal tissue may be ablated (606 and 607 respectively). The tissue damage or injury caused by the ablation may promote the healing or scarring process that may be adapted to promote the formation of tissue bridging. While the application of electrical energy is demonstrated, many other forms or combination of forms of ablation are adaptable to promote tissue bridging.

Following tissue ablation, the tissue securement device may be tightened and secured by methods described herein, as demonstrated in FIG. 18(C). The two portions of tissue 603, 604 have been apposed together with the ablated portions 606, 607 coming into at least partial contact with one another. Once the tissue apposition is secured and the ablated portions are at least partially in contact, the healing or scarring process may begin to grow the portions of tissue 603, 604 together. While two tissue portions are demonstrated in FIG. 18, embodiments of the present invention may comprise a plurality of tissue portions.

While FIG. 18 demonstrates an embodiment of damaging portions of tissue to promote tissue bridging, other embodiments are certainly possible. One such embodiment may comprise ablating the first and second portions of tissue prior to the tissue apposition device advancing a tissue securement device through the first or second portion of tissue. Another such embodiment comprises ablating the first and second portion of tissue after the tissue apposition means has been tightened and secured. Yet another embodiment comprises damaging the portions of tissue by a tissue ablation means incorporated into the tissue apposition device, whereby when the tissue is captured by the tissue apposition device, the tissue apposition device may ablate the collected tissue and cause tissue injury.

Alternate embodiments of the present invention are possible, wherein the application of ablation, such as electrical energy ablation, may be adapted to reinforce a tissue apposition. Electrical energy, or another appropriate form of ablation, may be applied to the tissue in order to promote a healing, growing, or scarring process. The application of ablation may be applied to one or more portions of tissue, whereby the ablation is conducted from the ablation source to the one or more portions of tissue via elements including, but not limited to, one or more tissue securement devices and one or more fixation agents. The one or more tissue securement devices or the one or more fixation agents may be comprised of a conductive material or have a coating of conductive material at least partially incorporated on or within the one or more tissue securement devices or one or more fixation agents. In such embodiments, when the ablation is conducted through a tissue securement device or fixation agent, the ablation may be discharged or applied to at least a portion of tissue that may be in contact with the tissue securement device or fixation agent. As a result, tissue damage may be applied to the at least a portion of tissue.

FIG. 19 illustrates one example of conducting ablation through a tissue securement device to cause tissue damage. In this example, the tissue securement device is embodied by suture material 632. Said suture material may be comprised of a material that is conductive of the ablation or coated with a material that is conductive of the ablation. The suture material 632, as demonstrated in FIG. 19A, is or has been advanced through two or more portions of tissue 630, 631 in a manner similar to methods and embodiments described herein. A source of ablation 633, such as an electrocautery catheter that may be used for the application of electrical energy, is positioned within the body cavity and may be placed in contact with the tissue securement device, shown in FIG. 19B. With the source of ablation at least partially in contact with the tissue securement device, the ablation may be applied. The ablation may be conducted through the tissue securement device and applied to one or more portions of tissue that are in contact with the tissue securement device. The application of ablation to one or more portions of tissue may cause tissue damage 634 to the tissue, which may promote a healing or scarring process. When new tissue or scar tissue has grown/formed in response to the tissue damage, the tissue may be tougher and/or more fibrous and therefore have a higher resistance to the tissue securement device pulling or tearing out of the one or more portions of tissue. By strengthening the resistance against tissue securement device migration out of the tissue, the embodiment reinforces the tissue apposition.

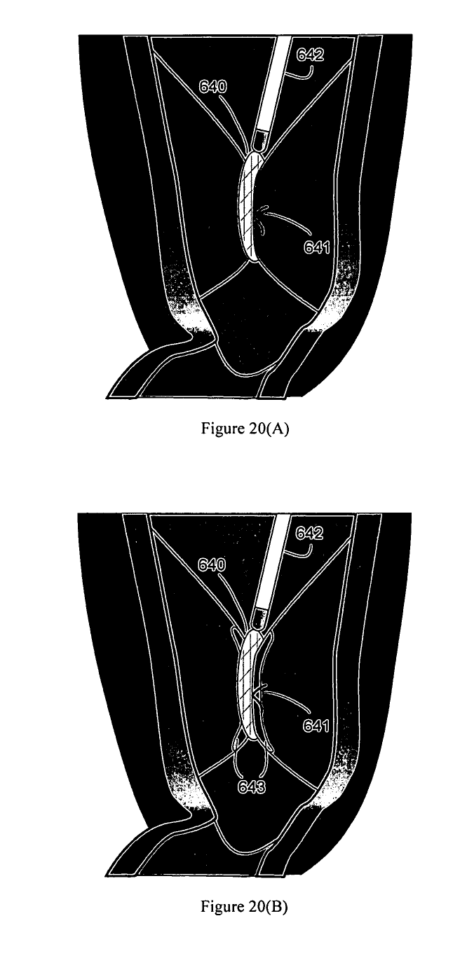

FIG. 20 demonstrates a further embodiment. In this embodiment, ablation may be conducted through a fixation agent to cause tissue damage. In this example, where the fixation agent may be embodied as a body of biocompatible fabric, said fixation agent may be comprised of a material that is conductive of the ablation or the fixation agent may be coated with a material that is conductive of the ablation.

A fixation agent is placed and secured within a tissue apposition in a manner similar to methods and embodiments described herein. With the fixation agent in place, as shown in FIG. 20A, an ablation source 642 is positioned within the body cavity and is placed at least partially in contact with the fixation agent 640. Ablation may then be applied by the ablation source and the ablation may be conducted through the fixation agent, whereby the ablation is applied to one or more portions of tissue in contact with the fixation agent. This is illustrated in FIG. 20B. The application of ablation may cause tissue damage 643 to the one or more portions of tissue, thereby promoting a growing scarring or healing process response. New tissue growth may infiltrate the fixation agent and fuse with one or more portions of other tissue. Tissue bridging may be formed, thereby reinforcing the tissue apposition and increasing its resistance to being separated.

Further embodiments of the present invention comprise the use of different forms or combinations of ablation. The application of one or more chemical substances, including but not limited to sodium morrhuate, to a portion of tissue may cause tissue damage and ablation. The chemical substance may be applied topically or injected below the surface of the portion of tissue. By performing tissue ablation with one or more chemical substances to one or more portions of tissue, said portions of tissue may be approximated and apposed with a tissue apposition device, whereby at least a portion of the ablated tissue is placed in contact with at least a portion of another ablated portion of tissue, such that tissue bridging may form. Such tissue bridging may reinforce the tissue apposition.

The use of mechanical means or mechanical abrasion may also be used to cause ablation in one or more portions of tissue. Examples of mechanical means or mechanical abrasion may include, but are not limited to, performing mucosal resection, or abrading the tissue with elements such as a rough texture member or with a brush-like device such as a cytology brush. The one or more portions of tissue may be abraded by one or more mechanical means and may be approximated and apposed with a tissue apposition device. When the healing or scarring process begins, the mechanically ablated portions of tissue may undergo tissue bridging to reinforce the tissue apposition.

Embodiments of the present invention may also use one of thermal ablation and cryo ablation. By exposing one or more portions of tissue to an extreme temperature, the mucosa and possibly the submucosa may be ablated. The one or more portions of tissue may be approximated and apposed within a tissue apposition, whereby the healing or scarring process may promote tissue bridging to reinforce the tissue apposition.

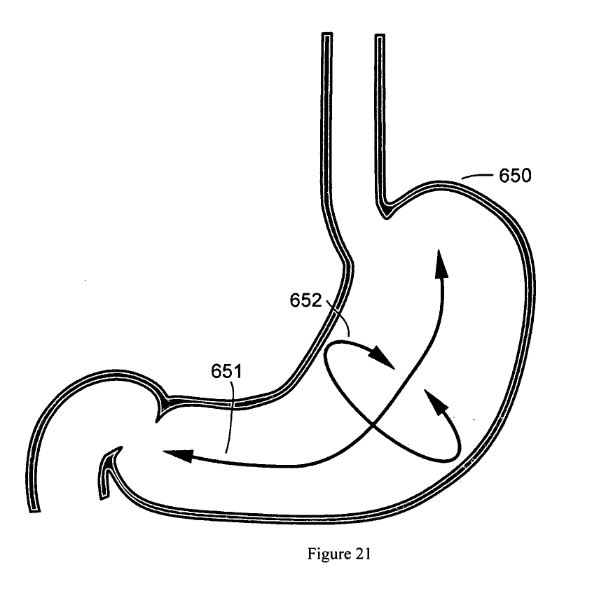

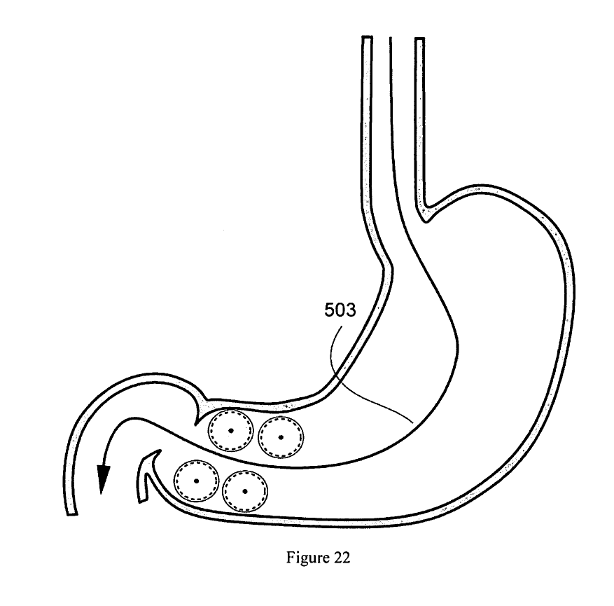

In certain embodiments of the present invention, a tissue apposition device, comprising at least one tissue securement device, may be positioned within the stomach to approximate and secure two or more portions of tissue together into a tissue apposition. The approximation and securing of two or more portions of tissue may be selected from methods described herein. The position of the portions of tissue comprised within the tissue apposition may be chosen from a plurality of sites within the stomach or organ system substantially adjacent to the stomach. Sites may include, but are not limited to, the stomach's fundus, cardia, body, antrum, and pylorus. The placement of one or more tissue appositions may inhibit or present forces that may oppose the forces exerted by the mechanical contractions of the stomach. The forces may be applied in manners including, but not limited to, longitudinal forces 651 and circumferential forces 652 within the stomach 650, both illustrated in FIG. 21. By opposing the forces exerted by the stomach, one or more of the stomach's functions may speed up or the function may slow down. For example, one or more tissue appositions may inhibit or slow down the stomach's peristalsis. Additionally, for example, one or more tissue appositions may inhibit the contractions of the antrum and/or pylorus, whereby the inhibition of the contractions cause the stomach's content to remain in the stomach for a longer period of time.

An embodiment of placing tissue appositions near the pylorus as illustrated in FIG. 22, whereby the placing tissue apposition may lengthen or elongate the pylorus or pylorus channel. By lengthening or elongating the pylorus or pylorus channel, the size of the stomach contents allowed to pass through the stomach and into the small intestine may be substantially reduced. In such cases, the stomach contents are held in the stomach for a longer period of time (delaying gastric emptying and possibly promoting satiety), whereby the stomach content may be further broken down. Lengthening or elongating the pylorus or pylorus channel in this manner may also shorten the antrum, which contributes to a portion of the grinding and/or propelling of stomach contents towards the pylorus. Shortening the antrum may reduce the grinding and propulsion forces in the stomach to further delay gastric emptying.

One function that may be altered as a result of one or more tissue appositions applying one or more forces to oppose one or more forces exerted by the stomach's mechanical contractions may include gastric transport. Particles of food and matter that enter the stomach (collectively referred to as stomach contents) are at least in part, mixed and transported through the stomach via stomach muscle contractions. Transportation may be accomplished by peristalsis or a peristalsis-like motion. By placing one or more tissue appositions within the stomach that apply one or more forces to oppose one or more forces exerted by the stomach's mechanical contractions, the gastric transport of stomach content may be altered.

Gastric emptying rate may be defined as the amount of time the stomach takes to transport stomach contents from the stomach into the intestines. By applying one or more forces that may oppose one or more forces exerted by the mechanical contractions of the stomach, the gastric emptying rate may increase—the stomach content may remain in the stomach longer. By keeping stomach contents within the stomach, the patient may maintain a sense of fullness and/or satiety longer and therefore potentially reduce the patient's food intake. The reduction in food intake may lead to weight loss.

Further embodiments of the invention may place one or more tissue apposition devices comprising one or more tissue securement devices, wherein the tissue apposition or method of placing the tissue apposition or the devices used in the creation of the tissue apposition alter the production of gastric secretions from portions of gastric secretion producing tissue. Said gastric secretion producing tissue may be comprised within the tissue apposition or in the substantial vicinity of the tissue apposition. Gastric secretions may include, but are not limited to hormones, stomach acid, and digestive enzymes.

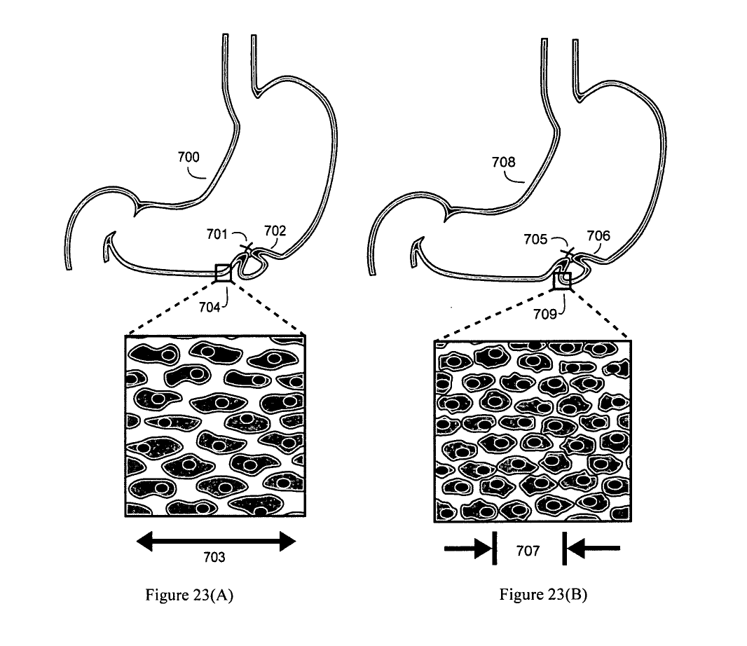

Embodiments of the present invention may place one or more tissue appositions within the stomach, wherein when the tissue securement device is tightened and possibly secured, the tightening exerts one or more forces on one or more portions of gastric secretion producing tissue. The one or more forces may be, for example longitudinal or circumferential in direction, as illustrated in FIG. 21. The one or more forces may alter the function of the one or more portions of gastric secretion producing tissue and alter the production of said gastric secretion. Such alterations may include the increase, decrease or cessation of gastric secretion production. Examples of forces exerted by a tissue securement device on a portion of gastric secretion producing tissue are illustrated in FIG. 23.

In FIG. 23A, a tissue securement device 701 is placed within a stomach 700, whereby the placement of the tissue securement device is such that a tissue apposition 702 is formed. As a result of the tissue apposition, tissue comprised within the tissue apposition or in the substantial vicinity of the tissue apposition 704 may be subjected to one or more forces as a result of the tissue apposition. The one or more forces may cause the gastric secretion producing tissue, such as hormone producing tissue, and/or the cells that make up the tissue to stretch 703, as indicated in the zoom-in box, whereby the stretching alters the gastric secretion production. An alternate example is illustrated in FIG. 23B. Again, a tissue securement device 705 is placed within a stomach 708 and a tissue apposition is formed. Gastric secretion producing tissue comprised within the tissue apposition or in the substantial vicinity of the tissue apposition 709 may be subjected to one or more forces exerted by the tissue apposition. Said forces may compress 707 the gastric secretion producing tissue and/or the cells that make up the tissue, as indicated by the zoom-in box of FIG. 23B, whereby the compression alters gastric secretion production.

Other embodiments exist, wherein the placing or advancing the tissue securement device through a portion of tissue may cause an alteration in the production of gastric secretion within gastric secretion producing tissue. Said alteration may be at least partially resulting from tissue damage caused by the placing or advancing of the tissue securement device or the presence of the securement device within the tissue.

By altering the hormone production of at least a portion of hormone producing tissue, the quantity of said hormone may increase or decrease within the patient's body. Embodiments that alter the hormone production of hormones that at least partially contribute to the patient's sensation of appetite or satiety may cause the patient to alter the amount of food that is eaten or taken in. This alteration in consumed food may cause the patient to lose weight as a result. Examples of such hormones that may at least partially contribute to the patient's sensation of appetite or satiety include, but are not limited to ghrelin, leptin, and adiponectin.

Embodiments of the present invention may alter the production of gastric secretions that at least partially contribute to the patient's ability to break down food particles within the stomach. Additionally, embodiments may alter the release of gastric secretions into the stomach, whereby causing delays in the gastric emptying rate of the patient and potentially promoting a feeling of satiety. Such a feeling of satiety may alter the amount of food that is eaten or taken in by the patient. This alteration in consumed food may cause the patient to lose weight as a result. Examples of such gastric secretions that may at least partially contribute to the patient's ability to break down particles in the stomach includes, but is not limited to gastric acid and digestive enzymes.

Certain embodiments of the present invention may inhibit the production of gastric produced hormones, such as ghrelin. In such embodiments, the advancement of the tissue securement device or the tightening of the tissue securement device may cause changes or promote inhibiting forces on endocrine cells within the stomach tissue. The changes or inhibiting forces on endocrine cells may include compression forces on the cells, stretching forces on the cells, disruption of intracellular space chemistry, disruption of ion transport in surrounding cells, or disruption of protein synthesis.

An embodiment of the present invention may comprise a tissue apposition device that may be positioned within the stomach cavity at the vicinity of the gastric fundus. Using one or more tissue securement devices, a tissue apposition may be created that may include at least a portion of the tissue in the vicinity of the gastric fundus. Given that endocrine cells residing in the vicinity of the gastric fundus are a main sourced of the production of hormones such as ghrelin, the tissue apposition may stretch, compress, or other wise alter the cellular environment which may negatively affect the protein synthesis of hormones such as ghrelin. As a result, the hormone production may be altered. The altered hormone production may cause a change in the satiety the patient experiences, thereby causing the individual to eat less food. The reduction in food intake may cause weight loss in the patient.

It is, of course, understood that modification of the present invention, in its various aspects, will be apparent to those skilled in the art. Additional method and device embodiments are possible, their specific features depending upon the particular application. For example, embodiments may be possible which comprise a tissue securement device using staples, pins, rods, wires, tags, or magnets to secure the tissue approximation. Additionally, multiple forms of ablation are possible including the combination of one or more forms of ablation to reinforce a tissue apposition.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

1. A method of promoting tissue adhesion to reinforce tissue apposition, the method comprising:
collecting a first tissue portion;
collecting a second tissue portion;
placing at least one tissue securement device through the first and second tissue portions;
tightening the at least one tissue securement device to approximate the first and second tissue portions; and
placing a fixation agent between the first and second tissue portions.

2. The method of claim 1, wherein the at least one tissue securement device includes suture material.

3. The method of claim 1, wherein the fixation agent includes a fabric comprising resorbable material.

4. The method of claim 3, wherein the fabric includes a plurality of interstices.

5. The method of claim 1, wherein the fixation agent includes a plurality of layers of material.

6. The method of claim 5, wherein the fixation agent includes one or more fenestrations that pass through the one or more layers of material.

7. The method of claim 5, wherein one or more of the plurality of layers of material is selected from the group consisting of biocompatible non-absorbable synthetic material, bioabsorbable or biodegradable synthetic material, non-absorbable material derived from natural sources, and bioabsorbable or biodegradable material derived from natural sources.

8. The method of claim 1, wherein the at least one tissue securement device is tightened after placing the fixation agent between the first and second tissue portions.

9. The method of claim 1, wherein the fixation agent reinforces and/or strengthens the tissue approximation between the first and second tissue portions.

10. The method of claim 1, wherein the fixation agent is adapted to promote tissue bridging between the first and second tissue portions.

11. The method of claim 1, wherein the first and second tissue portions are collected within a stomach.

12. The method of claim 11, wherein the first and second tissue portions are approximated to reduce the stomach.
